(19) United States
(12) Patent Application Publication
(10) Pub. No.: US 2023/0172500 A1
(43) Pub. Date: Jun. 8, 2023

(54) NON-INVASIVE TISSUE OXIMETRY DEVICE UTILIZING A MICRO-LASER

(71) Applicant: Edwards Lifesciences Corporation, Irvine, CA (US)

(72) Inventor: Muhammad Mujeeb-U-Rahman, Irvine, CA (US)

(21) Appl. No.: 18/162,143

(22) Filed: Jan. 31, 2023

Related U.S. Application Data
(63) Continuation of application No. PCT/US21/17244, filed on Feb. 9, 2021.
(60) Provisional application No. 63/060,472, filed on Aug. 3, 2020.

(51) Int. Cl. A61B 5/1455; A61B 5/00

(52) U.S. Cl. CPC A61B 5/14553; A61B 5/6814; A61B 2562/0238

(57) ABSTRACT

Disclosed is a non-invasive tissue oximetry device that is attachable to a patient's tissue to measure oxygen perfusion of the patient's tissue. The non-invasive tissue oximetry device includes: one or more micro-lasers to generate one or more optical signals; one or more detectors to receive the one or more optical signals; and a processor coupled to the one or more micro-lasers and detectors to measure oxygen perfusion of the tissue based upon the received one or more optical signals.

### CROSS REFERENCE

This application claims the benefit of PCT/US2021/017244, filed Feb. 9, 2021, which claims the benefit of U.S. Patent Application No. 63/060,472, filed Aug. 3, 2020, the entireties of each of which are hereby incorporated by reference.

### BACKGROUND

### Field

Embodiments of the invention relate generally to a non-invasive tissue oximetry device utilizing a micro-laser.

### Relevant Background

Non-invasive tissue oximetry is a revolutionary technology for the monitoring of oxygen perfusion of important biological tissues. It uses optical transmitters to generate signals at desired wavelengths that pass through biological tissue. A combination of detectors pick up the signals afterwards. A comparison of transmitted and received signals allows a device to estimate tissue properties including tissue perfusion. Current tissue oximetry devices are presently utilized for the monitoring of oxygen perfusion of certain biological tissues (e.g., the brain, or other tissues) by using optical transmitters to generate signals at desired wavelengths that pass through biological tissue. As examples, tissue oximetry devices may be used at a patient's forehead or at other body areas for monitoring oxygen perfusion.

In particular, the current designs of tissue oximetry devices utilize light emitting diodes (LEDs) for optical signal generation and optical detectors (e.g., photodiodes). Further, current designs of tissue oximetry devices utilize discrete electronics controllers for controlling the LEDs and optical detectors. Current designs of tissue oximetry devices utilize LEDs and discrete electronics controllers for optical signal generation, detection, and monitoring, which results in limitations of system performance.

For example, LEDs are economical but less efficient than other optical signal sources. As a particular example, high optical signal generation from LEDs requires a high-current operation. This increases the power dissipation in the circuit which increases temperature. Additionally, a significant limitation of high-powered LED operation is a shift in signal wavelength due to temperature increase. Because of this, system electronics becomes cumbersome (e.g., to compensate for temperature shifts due to thermal issues), thereby increasing system size, weight, and cost. Furthermore, in this type of system, it is not possible to operate the system in a continuous manner and gather a continuous waveform of the signal from the tissue under testing. Also, mostly discrete electronics are used in current devices, which adds to size and cost.

### SUMMARY

In one embodiment, a non-invasive tissue oximetry device is attachable to a patient's tissue to measure oxygen perfusion of the patient's tissue. The non-invasive tissue oximetry device includes: one or more micro-lasers to generate one or more optical signals; one or more detectors to receive the one or more optical signals; and a processor coupled to the one or more micro-lasers and detectors to measure oxygen perfusion of the tissue based upon the received one or more optical signals.

In one optional example, the oxygen perfusion of the tissue is measured in a continuous manner. In one optional example, the processor and one or more micro-lasers and detectors are integrated in the tissue oximetry device. In one optional example, the one or more micro-lasers include a vertical cavity surface emitting laser (VCSEL). In one optional example, the non-invasive tissue oximetry device is attachable to a patient's forehead to measure oxygen perfusion of the patient's brain or to a patient's muscle site to measure oxygen perfusion from the patient's muscle site. In one optional example, a display may be used to display the oxygen perfusion of the tissue. In one optional example, the non-invasive tissue oximetry device includes a rechargeable battery and a wireless transmitter to transmit data related to measured oxygen perfusion of the tissue. In one optional example, the one or more micro-lasers and a detector generate a photoplethysmogram (PPG) signal. In one optional example, the one or more micro-lasers include a plurality of micro-lasers that are switched in round-robin fashion to generate a PPG signal. In one optional example, the one or more detectors include an array detector to receive optical signals in synchronization with the micro-lasers to generate a PPG signal. It should be appreciated that the optional examples may be utilized independently from one another or in combination with one another.

In one embodiment, a method to measure a patient's tissue to measure oxygen perfusion of the patient's tissue is disclosed. The method comprises: attaching a non-invasive tissue oximetry device to the patient's tissue; controlling one or more micro-lasers to generate one or more optical signals; controlling one or more detectors to receive the one or more optical signals; and measuring oxygen perfusion of the tissue based upon the received one or more optical signals.

In one optional example, the oxygen perfusion of the tissue is measured in a continuous manner. In one optional example, the one or more micro-lasers and detectors are integrated in the tissue oximetry device. In one optional example, the one or more micro-lasers include a vertical cavity surface emitting laser (VCSEL). In one optional example, the non-invasive tissue oximetry device is attachable to a patient's forehead to measure oxygen perfusion of the patient's brain or to a patient's muscle site to measure oxygen perfusion from the patient's muscle site. In one optional example, a display may be used to display the oxygen perfusion of the tissue. In one optional example, the one or more micro-lasers and a detector generate a photoplethysmogram (PPG) signal. In one optional example, the one or more micro-lasers include a plurality of micro-lasers that are switched in round-robin fashion to generate a PPG signal. In one optional example, the one or more detectors include an array detector to receive optical signals in synchronization with the micro-lasers to generate a PPG signal. It should be appreciated that the optional examples may be utilized independently from one another or in combination with one another.

### BRIEF DESCRIPTION OF THE DRAWINGS

FIG. 1 is a diagram of a non-invasive tissue oximetry device, according to one optional example.

FIG. 2 is a diagram of a non-invasive tissue oximetry device, a controller, and a display, according to one optional example.

FIG. 3 is a diagram of a non-invasive tissue oximetry device and a wireless optical tissue monitoring device, according to one optional example.

FIG. 4 is a diagram of components of the non-invasive tissue oximetry device applied to a patient's tissue, according to one optional example.

FIG. 5A is a diagram showing an implementation to obtain a PPG-like signal utilizing micro-lasers and a detector, according to one optional example.

FIG. 5B is a diagram showing an implementation to obtain a PPG-like signal utilizing switching micro-lasers and a detector, according to one optional example.

FIG. 5C is a diagram showing an implementation to obtain a PPG-like signal utilizing micro-lasers and an array detector, according to one optional example.

### DETAILED DESCRIPTION

Embodiments of the invention generally relate to a non-invasive tissue oximetry device that is attachable to a patient's tissue to measure oxygen perfusion of the patient's tissue. The non-invasive tissue oximetry device may include: one or more micro-lasers to generate one or more optical signals; one or more detectors to receive the one or more optical signals; and a processor coupled to the one or more micro-lasers and detectors to measure oxygen perfusion of the tissue based upon the received one or more optical signals.

Aspects of the invention generally relate to a novel design for a non-invasive tissue oximetry device, which eliminates the limitations of current designs, resulting in better performance suitable for a wider variety of patients under wider use cases.

In particular, the non-invasive tissue oximetry device and methods, to be hereinafter described, generally relate to a novel design of a non-invasive tissue oximetry device that utilizes micro-lasers instead of LEDs, and that utilize integrated electronics, instead of discrete electronics, as utilized in current designs. Both of these features (use of micro-lasers and integrated electronics) help improve system size, cost, and performance (accuracy, richness of signal detected) compared to state-of-the-art systems currently used. Moreover, this design enables continuous operation which is essential to generate PPG waveform with rich information about the tissue beyond just measuring average oxygen concentration.

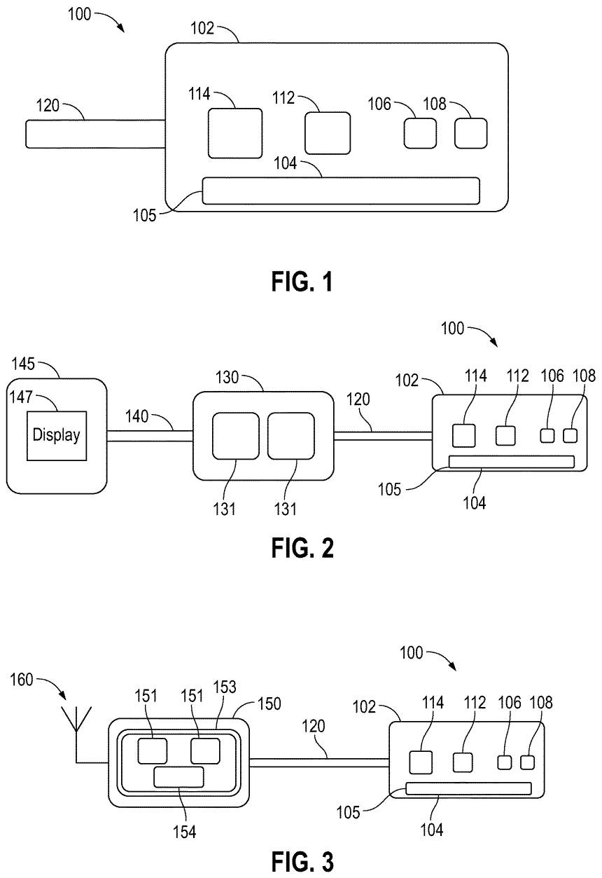

With reference to FIGS. 1 and 2, diagrams of a non-invasive tissue oximetry device 100, according one optional example, will be described. As can be seen with reference to FIG. 1, a non-invasive tissue oximetry device 100 that may be attachable to a patient's tissue to measure oxygen perfusion of the patient's tissue is disclosed. The non-invasive tissue oximetry device 100 may include: one or more micro-lasers 106 and 108 to generate one or more optical signals; one or more detectors 112 and 114 to receive the one or more optical signals; and a processor 104 coupled to the one or more micro-lasers 106, 108 and detectors 112, 114 that may be used to measure oxygen perfusion of the tissue based upon the received one or more optical signals.

As shown in FIG. 1, the non-invasive tissue oximetry device 100 may include micro-lasers 106, 108 to generate optical signals and detectors 112, 114 to receive the optical signals through a patient's tissue. It should be appreciated that any suitable number of micro-lasers to generate optical signals and that any suitable number of corresponding optical detectors to receive the optical signals may be utilized, FIG. 1 providing only an optional example. Further, in the optional example of FIG. 1, the processor 104 may be coupled to the micro-lasers 106, 108 and the detectors 112, 114 to measure oxygen perfusion of the tissue based upon the received one or more optical signals.

The non-invasive tissue oximetry device 100 may include a flexible housing package 102 to house the micro-lasers, detectors, processor and other electronic components. As an example, the flexible housing package 102 may be mounted/attached to a patient's forehead to measure oxygen perfusion of the patient's brain or to a patient's muscle site to measure oxygen perfusion from the patient's muscle site at any suitable location on the patient's body. As an example, the non-invasive tissue oximetry device 100 is attached such that the micro-lasers 106, 108 generate optical signals that pass through the patient's tissue for receipt by the detectors 112, 114, such that the processor 104 coupled to the one or more micro-lasers and detectors can measure oxygen perfusion of the tissue based upon the received one or more optical signals. As an example, the micro-lasers 106, 108 can generate optical signals at selectable wavelengths to perform the most optimal measurement of oxygen perfusion of the tissue. This may be used as a pulse oximetry method for monitoring and measuring a patient's oxygen saturation based upon received signals that are transmitted at particular wavelengths and comparison techniques that are applied to the received signals. Based upon optical signals received by the detectors 112, 114, the processor 104 can measure oxygen perfusion of the tissue based upon the received one or more optical signals. In one example, based upon optical signals received in a continuous manner by the detectors 112, 114, from the micro-lasers 106, 108, the processor 104 can measure oxygen perfusion of the tissue based upon the received one or more optical signals, which can thereafter be displayed. In one optional example, the oxygen perfusion of the tissue is measured in a continuous manner. In other optional examples, the oxygen perfusion of the tissue is measured in a semi-continuous manner.

As can be seen FIG. 1, the processor 104, the micro-lasers 106, 108 and detectors 112, 114 may all be integrated in the tissue oximetry device 100. As an example, all of the electronics 105 including the processor 104, memory, controllers, other electronic circuitry, etc., for the operation of the non-invasive tissue oximetry device 100 may be integrated and interconnected in the tissue oximetry device 100. Also, with brief reference to FIG. 2, the integrated tissue oximetry device 100 through an external interface cable 120 may be connected to a controller 130 that is further connected via an interface cable 140 to a patient monitoring device 145. The controller 130 may include suitable integrated electronics 131 for receiving, processing, and transmitting data (e.g., processor, memory, controllers, etc.). For example, the patient monitoring device 145 may be any type of computing device or medical electronic device that may read, collect, process, display, etc., physiological readings/data of a patient including a patient's oxygen saturation, as well as any other suitable physiological patient readings. Therefore, as an optional example, a display 147 of the patient monitoring device 145 may be used to display the oxygen perfusion of the tissue.

Additionally, with brief reference to FIG. 3, in one optional example, the tissue oximetry device 100 may be coupled by external interface cable 120 to a wireless optical tissue monitoring device 150. The wireless optical tissue monitoring device 150 may include suitable control electronics 151 for receiving, processing, and wirelessly transmitting data (e.g., processor, memory, controllers, transmitters, modems, etc.). Also, the wireless optical tissue monitoring device 150 may include a wireless charging coil 153 and a rechargeable battery 154, and a wireless communication transmitter link 160. Similar, to the previous implementation, wireless optical tissue monitoring device 150 may include suitable operation electronics 151 for receiving, processing, and wirelessly transmitting data to a patient monitoring device. Accordingly, the wireless transmitter link (e.g., including an antenna) 160 may wirelessly transmit data related to measured oxygen perfusion of the tissue, for example, to a patient monitoring device 145 (e.g., see FIG. 2). For example, the patient monitoring device 145 may be any type of computing device or medical electronic device that may read, collect, process, display, etc., physiological readings/data of a patient including patient's oxygen saturation, as well as any other suitable physiological patient readings. Therefore, as an optional example, a display 147 of the patient monitoring device 145 may be used to display the oxygen perfusion of the tissue. It should be appreciated that according to the previously described implementations, the tissue oximetry device 100 may be linked wirelessly or by a physical interface to a patient monitoring device. As should be appreciated, as described with reference to FIGS. 1-3, tissue oximetry device 100 may be suitably connected in a wired or wireless manner to a patient monitoring device. It should further be appreciated that tissue oximetry device 100 may be connected (wired or wirelessly) to the Internet or other networks such that the data from the tissue oximetry device 100 may be remotely measured and monitored.

Therefore, as previously described with reference to FIGS. 1-3, devices and methods generally relating to a novel design of a tissue oximetry device 100 that utilize micro-lasers, instead of LEDs, and that utilizes integrated electronics, instead of discrete electronics, have been shown and described. Both of these features (the use of micro-lasers and integrated electronics (e.g., processors, etc.)) help improve system size, cost, and performance (accuracy and richness of signal detected) compared to state-of-the-art systems that are used currently. The micro-lasers offer unique benefits in terms of optical efficiency, optical coherence, and a more focused and less scattered signal. This means overall power can be decreased, resulting in less thermal management issues and a more portable design.

In one optional example, the micro-lasers 106 and 108 of the tissue oximetry device 100 previously described may include vertical cavity surface emitting lasers (VCSELs). Such devices can provide an efficiency of 70%, a narrow beam angle (20°), with a perfect Gaussian profile, and narrow spectral width. Further, these devices are available in a variety of wavelengths ranging from red to near infrared/infrared wavelengths. In comparison, LEDs often have an efficiency of less than 50% (e.g., 20% typically), have much wider beams (e.g., viewing angles greater than 100°), and have less thermal stability and Non-Gaussian beam. As an example, during a measurement, a VCSEL with the same drive current can provide more than 60 times the on-axis power, when compared with an LED. Therefore, a VCSEL can provide significant improvements in system design, as compared to an LED. Additionally, the use of a VCSEL can reduce the complexity of system design by enabling lower-power drive electronics and a less involved detection circuit. Moreover, a single VCSEL or a combination of VCSELs, can be used to enable continuous operation, which can provide a more continuous waveform with richer information than that observed with a combination of LEDs requiring aggressive duty cycling for thermal management. It should be appreciated that a VCSEL is just one type of micro-laser that may be utilized. It should be appreciated that there are wide variety of other types of lasers or micro-lasers than may be used and that provide similar functionality.

In addition, with the use of micro-lasers (e.g., VCSELs), improvements can be made to system size, weight, and cost by using smaller and more integrated electronics than with prior LED implementations. As an example, field programmable analog array (FPAA) and field programmable gate array (FPGA) platforms may be used to minimize the development time and cost for mixed-mode operation. An FPGA with integrated analog capabilities may be used to decrease the complexity of electronics. Modern FPGAs come with significant existing capability (e.g., complete processor cores, I/O, memory, analog frontend) to enable such integrated design. For example, a combination of the field programmable device with small custom electronics may be optimally used. Further, customizable analog electronics may be used to provide many common analog processing and conditioning functions. Also, programmable analog devices may be used to reduce the size of the electronics. Additionally, a complete Application Specific Integrated Circuit (ASIC) could be utilized to provide necessary functions specific to the tissue oximetry device 100. Accordingly, the previously described electronics 105 including a processor 104 for implementing the operations of the tissue oximetry device 100 may be fully integrated and interconnected and provide significantly reduced design complexity and cost, as compared to previous implementations, enabled with a wide variety of different types of electronic implementations.

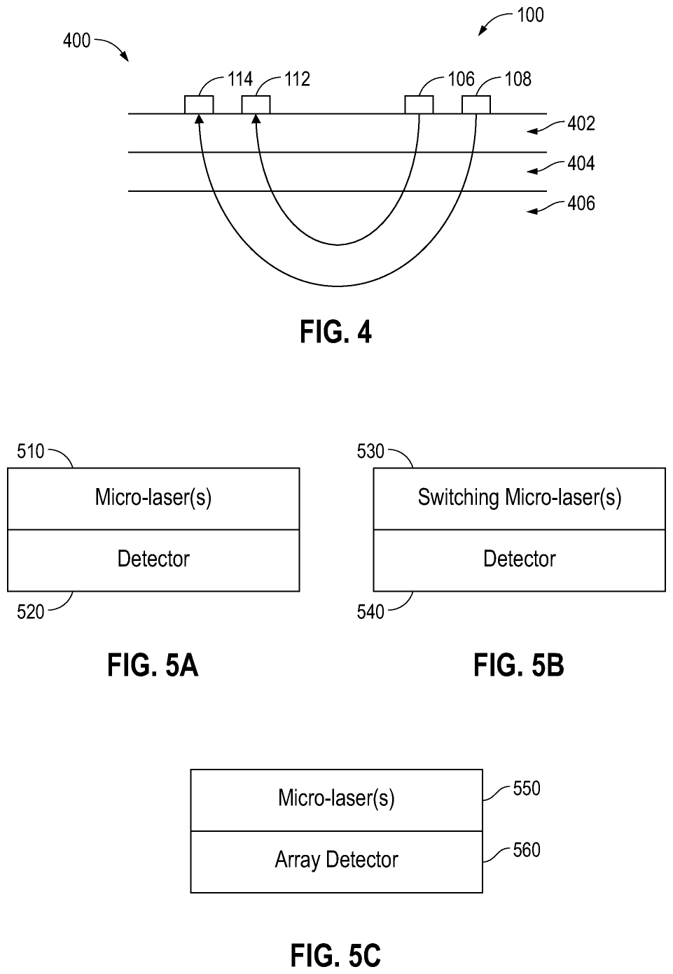

With additional reference to FIG. 4, an example patient tissue area 400 is provided as an example where the non-invasive tissue oximetry device 100 may be attached. As has been described, the tissue oximetry device 100 may include a flexible housing package to house the micro-lasers, detectors, processor and other electronic components, such that it can be attached to the tissue area 400 of a patient. As an example, the tissue oximetry device 100 may be mounted/attached to a patient's forehead to measure oxygen perfusion of the patient's brain or to a patient's muscle site to measure oxygen perfusion from the patient's muscle site at any suitable location on the patient's body.

As an optional example with reference to FIG. 4, the tissue oximetry device 100 may be attached to a patient's forehead. In this example, the micro-lasers 106, 108 generate optical signals that pass through the patient's forehead skin 402, skull 404, and brain 406, and are received by detectors 112, 114. As has been described, the processor coupled to the micro-lasers 106, 108 and the detectors 112, 114 can measure the oxygen perfusion of the tissue (e.g., the patient's brain) based upon the receipt of the optical signals form detectors 112, 114. As another optional example, the tissue oximetry device 100 may be attached to a patient's muscle site (e.g., at the patient's arm, flank, leg, etc.). In this example, the micro-lasers 106, 108 generate optical signals that pass through the patient's skin 402, muscle 404, and bone 406, and are received by detectors 112, 114. As has been described, the processor coupled to the micro-lasers 106, 108 and the detectors 112, 114 can measure the oxygen perfusion of the tissue (e.g., the patient's muscle) based upon the receipt of the optical signals form detectors 112, 114.

As has been described, the micro-lasers 106, 108 can generate optical signals at selectable wavelengths to perform the most optimal measurement of oxygen perfusion of the tissue. This may be used as a pulse oximetry method for monitoring and measuring a patient's oxygen saturation based upon received signals that are transmitted at particular wavelengths and comparison techniques that are applied to the received signals. Based upon optical signals received by the detectors 112, 114, the processor can measure oxygen perfusion of the tissue based upon the received one or more optical signals. In one example, based upon optical signals received in a continuous manner by the detectors 112, 114, from the micro-lasers 106, 108, the processor 104 can measure oxygen perfusion of the tissue based upon the received one or more optical signals, which can thereafter be displayed. In one optional example, the oxygen perfusion of the tissue is measured in a continuous manner. In other optional examples, the oxygen perfusion of the tissue is measured in a semi-continuous manner. As has been described, the patient's oxygen saturation of the tissue may be measured, processed, and displayed on a display device, as well as any other suitable physiological patient readings. It should be appreciated that the FIG. 4 example, of a forehead or muscle site, and example tissue layers, are merely examples, and the tissue oximetry device 100 may be attached at any suitable patient area for oxygen saturation measurement.

Therefore, a method to measure a patient's tissue to measure oxygen perfusion of the patient's tissue has been described that includes: attaching a non-invasive tissue oximetry device 100 to the patient's tissue 400; controlling one or more micro-lasers 106, 108 to generate one or more optical signals; monitoring one or more detectors 112, 114 to receive the one or more optical signals; and measuring oxygen perfusion of the tissue based upon the received one or more optical signals by the processor. It should be appreciated that dependent upon they type of measurement to be performed, the measurement of the oxygen perfusion of the tissue may be performed in a continuous or semi-continuous manner.

In other optional examples, methods by which a continuous or semi-continuous waveform that contains rich information about the pulsatility of local tissue perfusion can be obtained. In particular, by utilizing the previously described non-invasive tissue oximetry device 100 including micro-lasers, detectors, and a processor, a signal having approximately the same level of information as a common photoplethysmography (PPG) signal can be obtained. This PPG like signal can enable a wide range of applications that are not possible from a system that only gets a short duration signal from LEDs.

For example, with reference FIG. 5A, one example implementation to obtain a PPG-like signal waveform is to use one or more micro-lasers 510 in combination with a detector 520, and to use the receipt of the optical signals by the detector 520 through the tissue to generate a PPG-like signal waveform to obtain information regarding the pulsatility of local tissue perfusion. With additional reference to FIG. 5B, in one other example, an implementation to obtain a PPG-like signal is to use one or more switching micro-lasers 530 in combination with detector 540, and to combine the receipt of the optical signals by the detector 540 from these different sources to generate a PPG-like signal waveform to obtain information regarding the pulsatility of local tissue perfusion. If higher power operation is required, an array of micro-lasers can be used in a round-robin fashion. With additional reference to FIG. 5C, an array detector 560 can be used to detect signals from each micro-laser 550 and can be turned on in synchronization with the micro-lasers 550 to minimize the overlap in the signals to generate a PPG-like signal waveform to obtain information regarding the pulsatility of local tissue perfusion.

As has been described, aspects of the invention generally relate to a novel design for a non-invasive tissue oximetry device 100, which eliminates the limitations of current designs, resulting in better performance suitable for a wider variety of patients under wider use cases. As has been described, the non-invasive tissue oximetry device 100 and methods, previously described, generally relate to a novel design of a non-invasive tissue oximetry device that utilize micro-lasers instead of LEDs, and that utilizes integrated electronics, instead of discrete electronics, as utilized in current designs. Both of these features (use of micro-lasers and integrated electronics) help improve system size, cost, and performance (accuracy, richness of signal detected) compared to state-of-the-art systems currently used. In particular, as has been described, aspects of the invention significantly improves the performance of the tissue oximetry systems. It reduces the power budget of the driving circuitry and the sensitivity required for the detection circuitry. It also reduces the size and weight of the signal condition and processing circuitry and reduces overall package size and weight.

Further, the use of the micro-lasers offers unique benefits in terms of optical efficiency, optical coherence, and a more focused and less scattered signal. This means overall power can be decreased, resulting in less thermal management issues and a more portable design. Accordingly, the previously described electronics including a processor for implementing the operations of the tissue oximetry device may be fully integrated and interconnected and provide significantly reduced design complexity and cost, as compared to previous implementations, enabled by a wide variety of different types of electronic implementations, as previously described. Further, the use of lower power micro-lasers also enables more longer-term continuous operation of the system as compared to the use of LEDs. For example, one micro-laser may be used to obtain a continuous signal as it uses less power and hence can be kept on continuously (or turned off for a brief period and turned on for longer period).

Furthermore, embodiments of the invention are not limited to tissue oximetry but can be used in other applications where optical excitation and detection can be useful. The previously described implementations rely on transmission of the signal in the tissue and reflection of some of it that carries the information about tissue properties. Since multiple excitation sources (e.g., multiple VCSELs) are used in the system, more advanced signal processing can be used to extract more information from the system. For example, the internal pressure in the tissue can be measured by analyzing the signal at a different wavelength and its variation with the variations of tissue fluids (e.g., leakage in the tissue will raise fluid level and pressure and can be detected by observing its effect on different wavelengths).

It should be appreciated that the various previously described optional example implementations may be utilized independently from one another or in combination with one another. For example, the implementation of FIG. 1 including a non-invasive tissue oximetry device may be utilized independently or in combination with features of any of the other implementations including FIGS. 2, 3, and 5A-5C, in a suitable configuration. Also, the implementation of FIG. 2 including a non-invasive tissue oximetry device and an integrated control device utilizing a wired connection to patient monitoring device may be utilized independently or in combination with features of the wireless implementation of FIG. 3. Additionally, it should be appreciated that the implementations of FIGS. 5A-5C as to generating PPG-like signals based upon various detector configurations may be used independently from one another or in combination with one another, in a suitable configuration. Also, the implementations of FIGS. 1-3 may be used independently from the other implementations of FIGS. 5A-5C, or in combination with one or more of them, in a suitable configuration. Accordingly, it should be appreciated that a wide variety of the previously described optional examples may be utilized independently from one another or in combination with one or more of them, in a suitable configuration.

It should be appreciated that FIG. 1 illustrates a non-limiting example of a processor 104 included with integrated electronics 105 as well as micro-lasers and detectors to implement the functionality of the non-invasive tissue oximetry device 100, as previously described. As an example, the integrated electronics of the non-invasive tissue oximetry device may comprise a processor, a memory, and an input/output connected with a bus. Under the control of the processor, data may be received from an external source through the input/output interface and stored in the memory, and/or may be transmitted from the memory to an external destination through the input/output interface. The processor may process, add, remove, change, or otherwise manipulate data stored in the memory. Further, code may be stored in the memory. The code, when executed by the processor, may cause the processor to perform operations relating to data manipulation and/or transmission and/or any other possible operations. Similarly, the integrated electronics of the controller 130 and wireless device 150 may include processor and other integrated electronics to implement their previously described functionality.

It should be appreciated that aspects of the invention previously described may be implemented in conjunction with the execution of instructions by processors, circuitry, controllers, etc. As an example, a processor may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by circuitry, processors, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

The disclosure also includes the following clauses:

1. A non-invasive tissue oximetry device attachable to a patient's tissue to measure oxygen perfusion of the patient's tissue comprising:

one or more micro-lasers to generate one or more optical signals;

one or more detectors to receive the one or more optical signals; and

a processor coupled to the one or more micro-lasers and detectors to measure oxygen perfusion of the tissue based upon the received one or more optical signals.

2. The non-invasive tissue oximetry device of clause 1, wherein, oxygen perfusion of the tissue is measured in a continuous manner.
3. The non-invasive tissue oximetry device of any of the clauses 1-2, wherein, the processor and one or more micro-lasers and detectors are integrated in the tissue oximetry device.
4. The non-invasive tissue oximetry device of any of the clauses 1-3, wherein, the one or more micro-lasers include a vertical cavity surface emitting laser (VCSEL).
5. The non-invasive tissue oximetry device of any of the clauses 1-4, wherein, the non-invasive tissue oximetry device is attachable to a patient's forehead to measure oxygen perfusion of the patient's brain.
6. The non-invasive tissue oximetry device of any of the clauses 1-5, wherein, the non-invasive tissue oximetry device is attachable to a patient's muscle site to measure oxygen perfusion from the patient's muscle site.
7. The non-invasive tissue oximetry device of any of the clauses 1-6, further comprising, a display to display the oxygen perfusion of the tissue.
8. The non-invasive tissue oximetry device of any of the clauses 1-7, further comprising, a rechargeable battery and a wireless transmitter to transmit data related to measured oxygen perfusion of the tissue.
9. The non-invasive tissue oximetry device of any of the clauses 1-8, wherein, the one or more micro-lasers and a detector generate a photoplethysmogram (PPG) signal.
10. The non-invasive tissue oximetry device of any of the clauses 1-9, wherein, the one or more micro-lasers include a plurality of micro-lasers that are switched in round-robin fashion to generate a PPG signal.
11. The non-invasive tissue oximetry device of any of the clauses 1-10, wherein, the one or more detectors include an array detector to receive optical signals in synchronization with the micro-lasers to generate a PPG signal.
12. A method to measure a patient's tissue to measure oxygen perfusion of the patient's tissue comprising:

attaching a non-invasive tissue oximetry device to the patient's tissue;

controlling one or more micro-lasers to generate one or more optical signals;

monitoring one or more detectors to receive the one or more optical signals; and

measuring oxygen perfusion of the tissue based upon the received one or more optical signals.

13. The method of clause 12, wherein, oxygen perfusion of the tissue is measured in a continuous manner.
14. The method of any of the clauses 12-13, wherein, the one or more micro-lasers and detectors are integrated in the tissue oximetry device.
15. The method of any of the clauses 12-14, wherein, the one or more micro-lasers include a vertical cavity surface emitting laser (VCSEL).
16. The method of any of the clauses 12-15, wherein, the non-invasive tissue oximetry device is attachable to a patient's forehead to measure oxygen perfusion of the patient's brain.
17. The method of any of the clauses 12-16, wherein, the non-invasive tissue oximetry device is attachable to a patient's muscle site to measure oxygen perfusion from the patient's muscle site.
18. The method of any of the clauses 12-17, further comprising, a display to display the oxygen perfusion of the tissue.
19. The method of any of the clauses 12-18, wherein, the one or more micro-lasers and a detector generate a photoplethysmogram (PPG) signal.
20. The method of any of the clauses 12-19, wherein, the one or more micro-lasers include a plurality of micro-lasers that are switched in round-robin fashion to generate a PPG signal.
21. The method of any of the clauses 12-20, wherein, the one or more detectors include an array detector to receive optical signals in synchronization with the micro-lasers to generate a PPG signal.

What is claimed is:

1. A non-invasive tissue oximetry device attachable to a patient's tissue to measure oxygen perfusion of the patient's tissue comprising:
one or more micro-lasers to generate one or more optical signals;
one or more detectors to receive the one or more optical signals;
a processor coupled to the one or more micro-lasers and detectors to measure oxygen perfusion of the tissue based upon the received one or more optical signals;
wherein, the non-invasive tissue oximetry device is attachable to a patient's muscle site to measure oxygen perfusion from the patient's muscle site; and
wherein, the one or more micro-lasers include a plurality of micro-lasers that are switched in round-robin fashion to generate a PPG signal.

2. A non-invasive tissue oximetry device attachable to a patient's tissue to measure oxygen perfusion of the patient's tissue comprising:
one or more micro-lasers to generate one or more optical signals;
one or more detectors to receive the one or more optical signals; and
a processor coupled to the one or more micro-lasers and detectors to measure oxygen perfusion of the tissue based upon the received one or more optical signals.

3. The non-invasive tissue oximetry device of claim 2, wherein oxygen perfusion of the tissue is measured in a continuous manner.

4. The non-invasive tissue oximetry device of claim 2, wherein the processor and one or more micro-lasers and detectors are integrated in the tissue oximetry device.

5. The non-invasive tissue oximetry device of claim 2, wherein the one or more micro-lasers include a vertical cavity surface emitting laser (VCSEL).

6. The non-invasive tissue oximetry device of claim 2, wherein the non-invasive tissue oximetry device is attachable to a patient's forehead to measure oxygen perfusion of the patient's brain.

7. The non-invasive tissue oximetry device of claim 2, wherein the non-invasive tissue oximetry device is attachable to a patient's muscle site to measure oxygen perfusion from the patient's muscle site.

8. The non-invasive tissue oximetry device of claim 2, further comprising, a display to display the oxygen perfusion of the tissue.

9. The non-invasive tissue oximetry device of claim 2, further comprising comprising, a rechargeable battery and a wireless transmitter to transmit data related to measured oxygen perfusion of the tissue.

10. The non-invasive tissue oximetry device of claim 2, wherein the one or more micro-lasers and the one or more detectors generate a photoplethysmogram (PPG) signal.

11. The non-invasive tissue oximetry device of claim 10, wherein the one or more micro-lasers include a plurality of micro-lasers that are switched in round-robin fashion to generate the PPG signal.

12. The non-invasive tissue oximetry device of claim 11, wherein the one or more detectors include an array detector to receive optical signals in synchronization with the micro-lasers to generate the PPG signal.

13. A method to measure a patient's tissue to measure oxygen perfusion of the patient's tissue comprising:
attaching a non-invasive tissue oximetry device to the patient's tissue;
controlling one or more micro-lasers to generate one or more optical signals;
monitoring one or more detectors to receive the one or more optical signals; and
measuring oxygen perfusion of the tissue based upon the received one or more optical signals.

14. The method of claim 13, wherein, oxygen perfusion of the tissue is measured in a continuous manner.

15. The method of claim 14, wherein the one or more micro-lasers and detectors are integrated in the tissue oximetry device.

16. The method of claim 15, wherein the one or more micro-lasers include a vertical cavity surface emitting laser (VCSEL).

17. The method of claim 16, wherein the non-invasive tissue oximetry device is attachable to a patient's forehead to measure oxygen perfusion of the patient's brain.

18. The method of claim 16, wherein the non-invasive tissue oximetry device is attachable to a patient's muscle site to measure oxygen perfusion from the patient's muscle site.

19. The method of claim 18, further comprising, a display to display the oxygen perfusion of the tissue.

20. The method of claim 19, wherein, the one or more micro-lasers and the one or more detectors generate a photoplethysmogram (PPG) signal.

21. The method of claim 20, wherein, the one or more micro-lasers include a plurality of micro-lasers that are switched in round-robin fashion to generate the PPG signal.

22. The method of claim 21, wherein, the one or more detectors include an array detector to receive optical signals in synchronization with the micro-lasers to generate a PPG signal.
